(19) United States
(12) Patent Application Publication
(10) Pub. No.: US 2023/0192702 A1
(43) Pub. Date: Jun. 22, 2023

(54) PROCESS FOR THE PREPARATION OF VERDIPERSTAT

(71) Applicant: Biohaven Therapeutics Ltd., New Haven, CT (US)

(72) Inventors: Yadagiri R. PENDRI, Storrs, CT (US); Srinivasa KATHAM, Hyderabad (IN); Dhanaraj Reddy CHILAKALA, Hyderabad (IN); Chendrasekhar KEDAS, Hyderabad (IN); Srinivasa Rao KANDIYALA, Hyderabad (IN); Sreenivas MENDE, Hyderabad (IN)

(21) Appl. No.: 17/922,409

(22) PCT Filed: May 5, 2021
(86) PCT No.: PCT/US21/30766

§ 371 Date: Oct. 31, 2022

Related U.S. Application Data
(60) Provisional application No. 63/021,041, filed on May 6, 2020.

(51) Int. Cl. C07D 487/04

(52) U.S. Cl. CPC C07D 487/04

(57) ABSTRACT

An improved process for preparing verdiperstat is disclosed. The process includes the steps of reacting a compound having formula

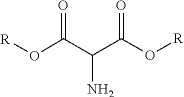

or a salt thereof, wherein R is the same or different and is each independently a C1-05 alkyl with 3-(dimethylamino)acrylonitrile to obtain a compound having formula

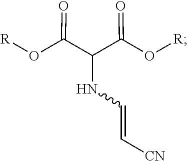

and converting the compound having formula

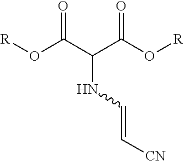

to verdiperstat.

### CROSS-REFERENCE TO RELATED APPLICATIONS

This application claims priority to U.S. Provisional Application No. 63/021,041 filed May 6, 2020 and all the benefits accruing therefrom under 35 U.S.C. § 119, the content of which is incorporated herein in its entirety by reference.

### FIELD OF THE INVENTION

The present invention relates to a process for the preparation of 1-(2-isopropoxyethyl)-2-thioxo-1,2,3,5-tetrahydro-pyrrolo[3,2-d]pyrimidin-4-one (verdiperstat).

### BACKGROUND OF THE INVENTION

Verdiperstat (1, also known as BHV-3241 or AZD3241) is a potential first-in-class, oral, brain-penetrant, irreversible inhibitor of myeloperoxidase (MPO), an enzyme that acts as a key driver of pathological oxidative stress and inflammation in the brain.

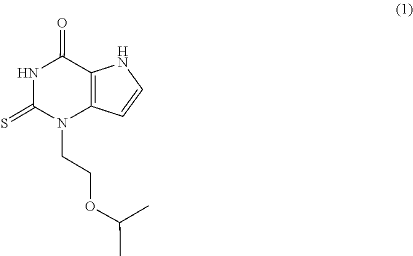 [SMILES: CC(C)OCCn1c(=S)[nH]c(=O)c2[nH]ccc21]

MPO is believed to play an important role in multiple neurodegenerative diseases, because it increases oxidative stress and inflammation levels in the brain. Inhibiting MPO is believed to ameliorate these pathological mechanisms, which are strongly implicated in the onset and progression of multiple system atrophy (MSA) and amyotrophic lateral sclerosis (ALS), progressive and fatal neurodegenerative diseases with limited treatment options.

Results from a Phase 2 trial in MSA showed evidence of target engagement and favorable trends over 12 weeks on the Unified MSA Rating Scale, an exploratory clinical outcome measure. Verdiperstat has received orphan drug designation for the treatment of MSA from the United States Food and Drug Administration (FDA) Office of Orphan Products Development as well as the European Commission upon recommendation from the European Medicines Agency's Committee for Orphan Medicinal Products. The FDA has subsequently issued a May Proceed Letter to initiate Phase 3 clinical trial of verdiperstat in MSA. Verdiperstat has also been selected as a candidate for a platform trial collaboration for ALS.

The ongoing clinical trials of verdiperstat require large quantities of pure chemical material. For purposes of large-scale production, there is a need for high-yielding synthesis of verdiperstat that is efficient, safe, and cost-effective.

### SUMMARY OF THE INVENTION

The present invention is directed to the process for the preparation of verdiperstat that is efficient, safe, and cost-effective.

In an embodiment, a process for preparing verdiperstat is provided. The process includes the steps of:

reacting a compound having formula

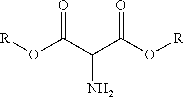

or a salt thereof, wherein R is independently a C1-C5 alkyl, with 3-(dimethylamino)acrylonitrile to obtain a compound having formula

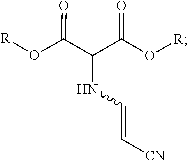

and

converting the compound having formula

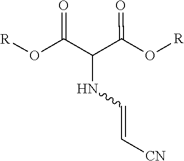

to verdiperstat.

In another embodiment, a process for preparing verdiperstat may include:

Step (a): reacting cyanoacetic acid with N, N-dimethylformamide dimethyl acetal to obtain 3-(dimethylamino)acrylonitrile;

Step (b): reacting a compound having formula

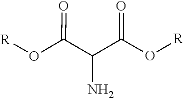

or a salt thereof, wherein R is the same or different and is each independently a C1-C5 alkyl, with 3-(dimethylamino)acrylonitrile to obtain a compound having formula

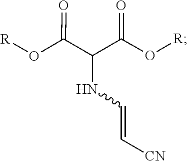

Step (c): converting the compound having formula

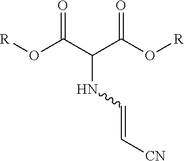

to a compound having formula

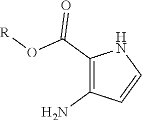

or a salt thereof;

Step (d): reacting the compound having formula

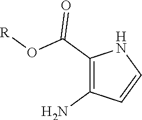

or a salt thereof with a compound having formula

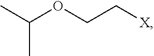

wherein X is a leaving group to obtain a compound having formula

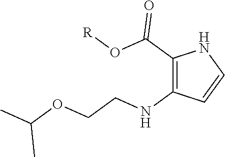

or a salt thereof;

Step (e): reacting the compound having formula

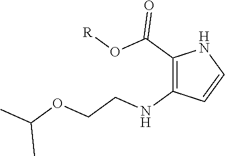

or a salt thereof with a compound having formula

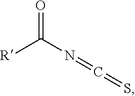

wherein R′ is H or an organic group, to obtain a compound having formula

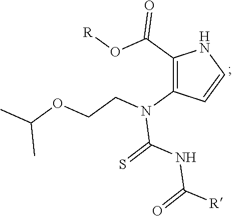

and

Step (f): converting the compound having formula

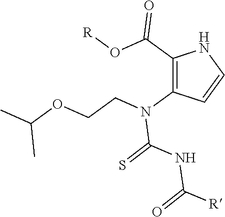

to verdiperstat.

In another embodiment, a composition for the preparation of verdiperstat is provided. The composition may include:

a compound having formula

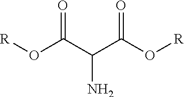

or a salt thereof, wherein R is the same or different and is each independently a C1-C5 alkyl; and

3-(dimethylamino)acrylonitrile.

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —CI, —Br, —I), a hydroxy group (—OH), an amino group (—NH₂), a carboxyl group (—CO₂H), a substituted or unsubstituted C1-C10 amine group, a nitro group (—NO₂), a C1-C10 alkyl group, a C3-C10 cycloalkyl group, a C6-C12 aryl group, a C1-C10 alkoxy group, a C1 to C10 trifluoroalkyl group such as a trifluoromethyl group (—CF₃) and the like, or a cyano group (—CN) instead of at least one hydrogen of a substituting group or compound.

Additional aspects will be set forth in part in the description which follows and, in part, will be apparent from the description.

The starting materials useful for making the pharmaceutical compositions of the present invention are readily commercially available or can be prepared by those skilled in the art.

Existing synthetic approaches to verdiperstat, including 3-amino-1H-pyrrole-2-carboxylate as an intermediate, have substantial shortcomings which preclude them from being implemented on a large scale. One of them is shown in Reaction Scheme 1 below:

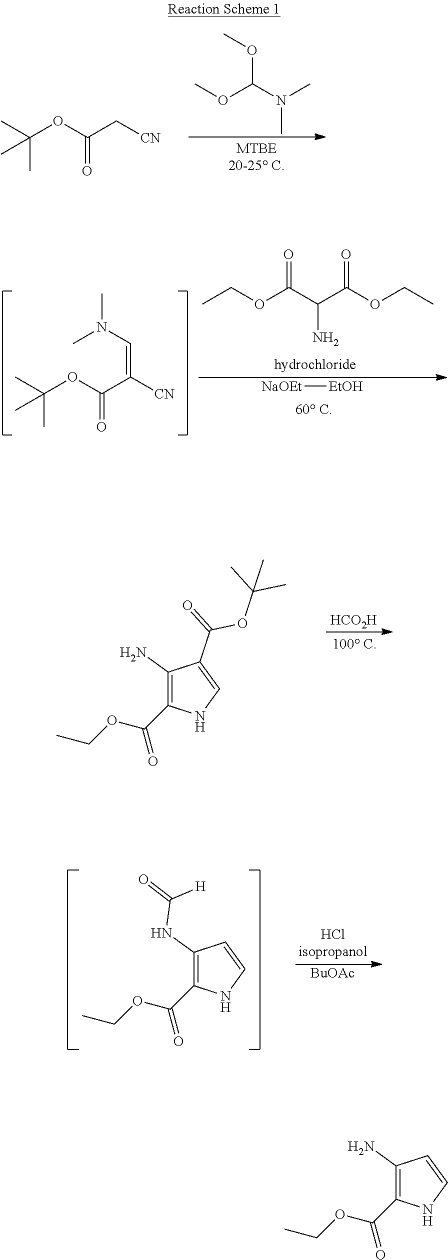

This approach requires four steps with only one isolated intermediate, thus making purity control difficult. The synthesis employs an expensive protecting group, and after formation of the core pyrrole ring, two distinct unit operations are still needed to obtain ethyl 3-amino-1H-pyrrole-2-carboxylate.

The other approach is shown in Reaction Scheme 2 below (Wu X. et al. Chemical Biology and Drug Design 2011, 78, 932-940):

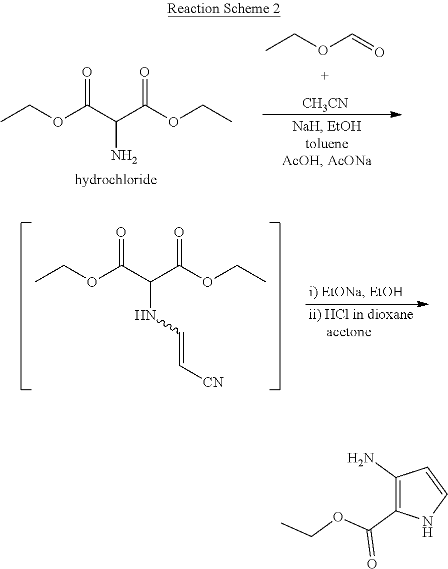

Although this route appears to be short, it utilizes highly hazardous chemicals (sodium hydride, 1,4-dioxane) making it difficult to implement the transformation on a process scale. The synthetic steps in this approach also have little reproducibility.

The present invention is directed to the process for the preparation of verdiperstat that is efficient, safe, and cost-effective. The process is convergent, utilizes no protecting groups, and involves non-hazardous chemicals. Thus, the inventive process can be easily adopted to a large-scale synthesis of verdiperstat.

In an embodiment, a process for preparing verdiperstat is provided. The process includes the steps of:

reacting a compound having formula

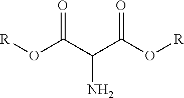

or a salt thereof, wherein R is independently a C1-C5 alkyl, with 3-(dimethylamino)acrylonitrile to obtain a compound having formula

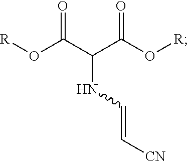

and

converting the compound having formula

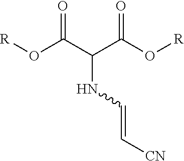

to verdiperstat.

In the compound having formula

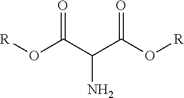

or a salt thereof, R may be a linear or branched C1-C5 alkyl group. For example, R may be methyl, ethyl, n-propyl, iso-propyl, n-butyl, iso-butyl, sec-butyl, tert-butyl, n-pentyl, iso-pentyl, sec-pentyl, tert-pentyl, or neo-pentyl group. Each of these group may be substituted or unsubstituted.

The step of reacting a compound having formula

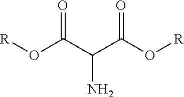

or a salt thereof, with 3-(dimethylamino)acrylonitrile may be conducted in the presence of an acid. The acid may be an inorganic (mineral) acid or organic acid. The organic acid may be a C1-C20 carboxylic acid, which may be linear or branched, substituted or unsubstituted. Examples of carboxylic acids may include formic acid, acetic acid, propionic acid, butyric acid, valeric acid, but are not limited thereto.

The step of converting the compound having formula

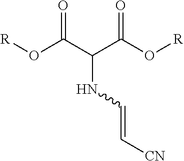

to verdiperstat may include:

converting the compound having formula

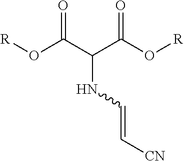

to a compound having formula

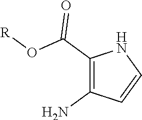

or a salt thereof; and converting the compound having formula

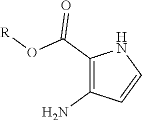

or a salt thereof to verdiperstat.

The step of converting the compound having formula

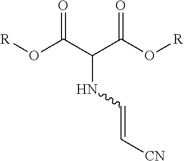

to a compound having formula

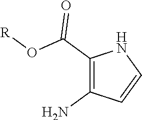

or a salt thereof may be conducted in the presence of a base. The base may be a metal hydroxide, metal alkoxide, a metal carbonate, a metal phosphate. A metal may be an alkali metal or alkaline earth metal. Examples of bases may include lithium alkoxide, sodium alkoxide, potassium alkoxide, rubidium alkoxide, or cesium alkoxide, but are not limited thereto.

The step of converting the compound having formula

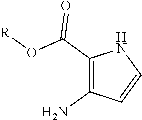

or a salt thereof to verdiperstat may include reacting the compound having formula

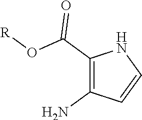

pr a salt thereof with a compound having formula

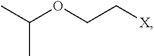

wherein X is a leaving group to obtain a compound having formula

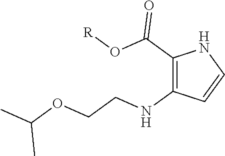

or a salt thereof. The leaving group may be a halogen or an organic sulfonate group. Examples of the organic sulfonate group may include tosylate, mesylate, or triflate, but are not limited thereto.

The step of reacting the compound having formula

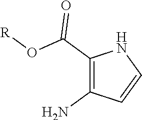

or a salt thereof with a compound having formula

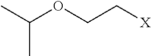

may be conducted in the presence of a base, and optionally, a metal iodide. The base may be a salt of an inorganic acid, and the metal may be an alkali metal. The salt of the inorganic acid may be a carbonate, a hydrocarbonate, a sulfate, a hydrosulfate, a phosphate, a hydrophosphate, or a dihydrophosphate.

The step of converting the compound having formula

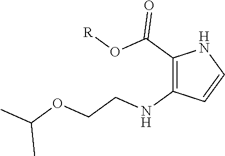

or a salt thereof to verdiperstat may further include reacting the compound having formula

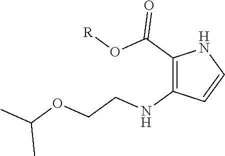

or a salt thereof with a compound having formula

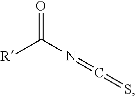

wherein R′ is H or an organic group, to obtain a compound having formula

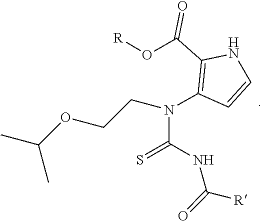

In the compound having formula

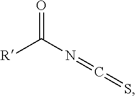

R′ may be an aliphatic organic group (for example, a C1-C30 alkyl group), a heteroaliphatic organic group (for example, a C1-C30 heteroalkyl group), an aromatic organic group (for example, a C6-C30 aryl group), or a heteroaromatic organic group (for example, a C3-C30 heteroaryl group), each of which may be substituted or unsubstituted.

The step of converting the compound having formula

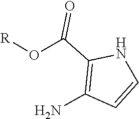

or a salt thereof to verdiperstat may further include converting the compound having formula

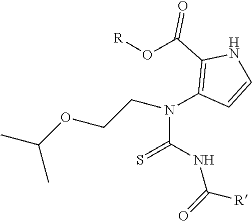

to verdiperstat.

The step of converting the compound having formula

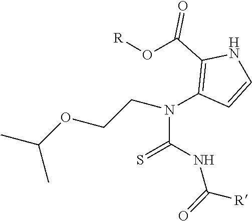

to verdiperstat may be conducted in the presence of a base. Examples of bases may include a metal hydroxide, a metal alkoxide, a metal carbonate, or a metal phosphate, but are not limited thereto.

The step of reacting a compound having formula

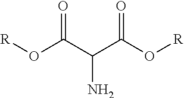

or a salt thereof, wherein R is a C1-C5 alkyl, with 3-(dimethylamino)acrylonitrile may be preceded by reacting cyanoacetic acid with N, N-dimethylformamide dimethyl acetal to obtain 3-(dimethylamino)acrylonitrile.

In another embodiment, a process for preparing verdiperstat may include:

Step (a): reacting cyanoacetic acid with N, N-dimethylformamide dimethyl acetal to obtain 3-(dimethylamino)acrylonitrile;

Step (b): reacting a compound having formula

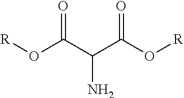

or a salt thereof, wherein R is the same or different and is each independently a C1-C5 alkyl, with 3-(dimethylamino)acrylonitrile to obtain a compound having formula

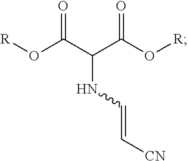

Step (c): converting the compound having formula

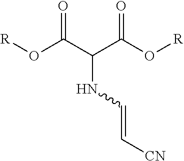

to a compound having formula

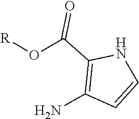

or a salt thereof;

Step (d): reacting the compound having formula

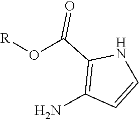

or a salt thereof with a compound having formula

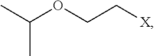

wherein X is a leaving group to obtain a compound having formula

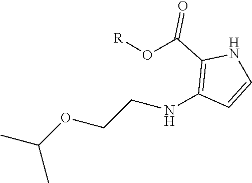

or a salt thereof;

Step (e): reacting the compound having formula

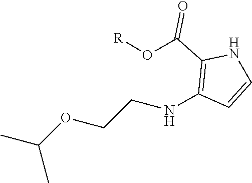

or a salt

thereof with a compound having formula

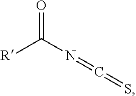

wherein R′ is H or an organic group, to obtain a compound having formula

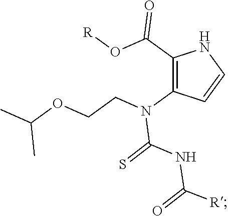

and

Step (f): converting the compound having formula

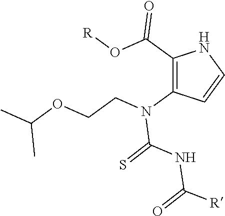

to verdiperstat.

In another embodiment, a composition for the preparation of verdiperstat is provided. The composition may include:

a compound having formula

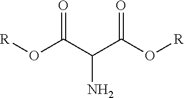

or a salt thereof, wherein R is the same or different and is each independently a C1-C5 alkyl; and

3-(dimethylamino)acrylonitrile.

The composition may further include a solvent, an acid, or a combination thereof. The solvent may be a hydrocarbon solvent, a halogenated hydrocarbon solvent, or a combination thereof. The solvent is not particularly limited and can be selected to sufficiently solubilize the reaction components.. The acid may be an inorganic (mineral) acid or an organic acid. The organic acid may be a C1-C20 carboxylic acid, which may be linear or branched, substituted or unsubstituted. Examples of carboxylic acids may include formic acid, acetic acid, propionic acid, butyric acid, valeric acid, but are not limited thereto.

Provided also is a composition including verdiperstat and a compound having formula

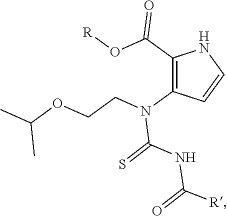

wherein R′ is H or an organic group. In an embodiment, the compound having formula

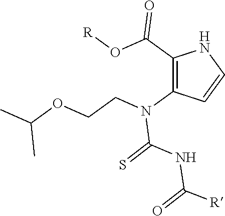

may be present in an amount of 0.2 percent by weight or greater based on the total weight of the composition. In another embodiment, the compound having formula

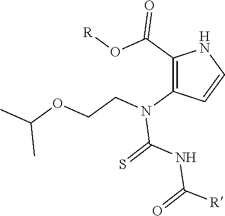

may be present in an amount of less than 0.2 percent by weight based on the total weight of the composition.

The invention is further illustrated by the following non-limiting example.

### EXAMPLE

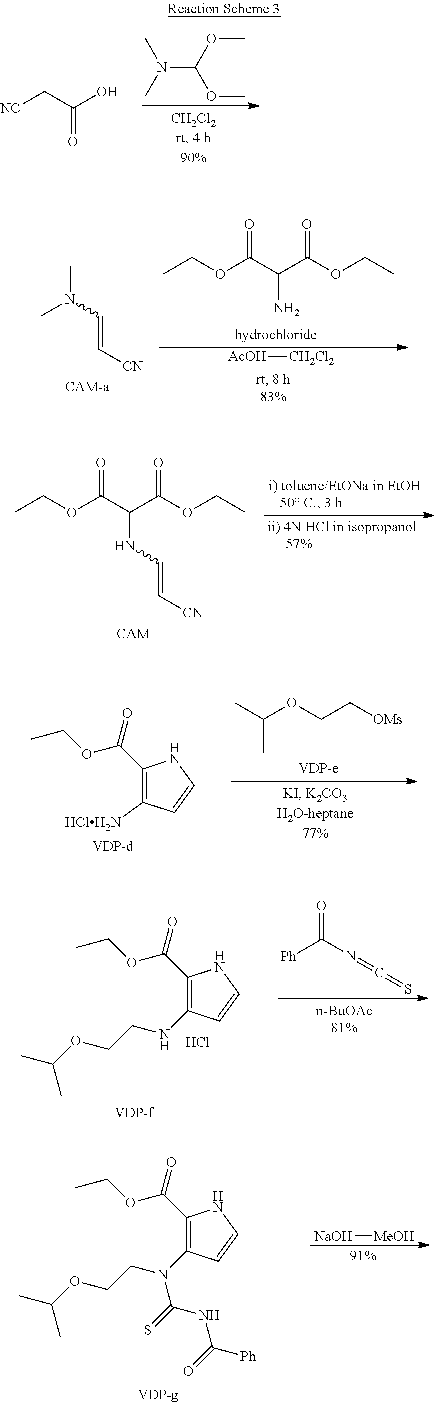

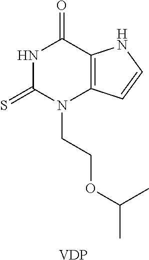 [SMILES: CC(C)OCCn1c(=S)[nH]c(=O)c2[nH]ccc21 ; "VDP"]

STEP (a): Synthesis of 3-(dimethylamino)acrylonitrile (CAM-a)

To a stirred solution of cyanoacetic acid (100 g, 1.17 mole) in dichloromethane (500 mL) was added N, N-dimethylformamide dimethyl acetal (140 g, 1.17 mole) at 30±5° C. and the reaction mixture was stirred at 30±5° C. for 12 hours. The completion of reaction was monitored by GC (cyanoacetic acid content NMT 5%). After completion, the reaction mass was concentrated under vacuum to afford 113 g of brown colored 3-(dimethylamino) acrylonitrile as a liquid which was taken to the next step without further purification.

Result: Input: 100 g, Output: 113 g, crude yield: 100%, assay yield: 90%, GC purity: 91.24%.

¹ NMR (400 MHz, CDCI₃): δ 6.93 (d, 1H, J=13.6 Hz), 3.67 (d, 1H, J=13.6 Hz), 2.86 (bs, 6H). Mass m/z (%): [M+H]⁺: 97.1.

STEP (b): Synthesis of diethyl 2-((2-cyanovinyl) amino) malonate (CAM)

Method A: To a stirred solution of 2-aminodiethylmalonate hydrochloride (150 g, 0.708 mmol) and 3-(dimethylamino) acrylonitrile (113 g, 1.17 mol) in dichloromethane (500 mL) at 30±5° C. was added acetic acid (100 g, 1.66 mol) dropwise and the reaction mixture was maintained at the same temperature for 12 h. The completion of reaction was monitored by GC (aminodiethyl malonate hydrochloride content NMT 5%). Then the reaction mixture was washed with water twice (2×100 mL) and the aqueous layer was extracted with dichloromethane (100 mL). The combined organic layer was washed with 5% aq. citric acid solution (1×100 mL) followed by 10% sodium bicarbonate solution (1×100 mL) followed with water (100 mL). The organic layer was concentrated and co-distilled with toluene (100 mL). The resulting brown coloured semi-solid (150 g) was taken to the next step without further purification.

Result: Input: 150 g, Output: 150 g, Crude yield: 93.7%, Assay yield: 83%, Purity: 92.7%.

Method B: To a stirred solution of 2-aminodiethylmalonate hydrochloride (150 g, 0.708 mmol) and 3-(dimethylamino) acrylonitrile (113 g, 1.17 mol) in 1,4-dioxane (300 mL) then heated to at 50±5° C. for 4 hours. The completion of reaction was monitored by GC (aminodiethyl malonate hydrochloride content NMT 5%). After completion of reaction add water (300 mL) in to the reaction mixture at 25±5 ° C., then filter the precipitated solid and washed with water (50 mL) and dried under vacuum at below 50±5° C. The resulting brown coloured solid (120 g) was taken to the next step without further purification.

Result: Input: 150 g, Output: 120 g, Crude yield: 75%, Assay yield: 90%, Purity: 95.0%.

¹NMR (400 MHz, CDCl₃): δ 7.03 (dd, 1H, J=7.2, 14.0 Hz), 5.56 (br t, 1H, J=6.0 Hz), 4.48 (d, 1H, J =6.8 Hz), 4.34- 4.26 (m, 4H), 4.0 (d, 1H, J=14.4 Hz), 1.31 (t, 6H, J=7.2).

Mass m/z (%): [M+H]⁺: 227.18.

STEP (c): Synthesis of Ethyl 3-amino-1H-pyrrole-2-carboxylate hydrochloride (VDP-d)

A stirred solution of sodium ethoxide (60.1 g, in 500 mL ethanol) was added to a stirred suspension of diethyl 2-((2-cyanovinyl) amino) malonate (100 g (assay: 83%), 0.442 mol) in anhydrous toluene (1.6 L) at 55° C. over a period of 10 minutes. The resulting suspension was stirred at 55 to 60° C. for 5 hours to complete the reaction. After completion of the reaction (the reaction mass was monitored by HPLC, the content of diethyl 2-((2-cyanovinyl) amino) malonate is NMT 5%), the reaction mixture was cooled to 25±5° C., and the reaction mixture pH was slowly adjusted to 7 to 8 by adding acetic acid (110 g). The toluene and ethanol solution was then concentrated under reduced pressure at below 55° C. The resulting crude residue was diluted with ethyl acetate-water (2:1; 150 mL) and stirred for 30 minutes. The organic layer was separated and the aqueous layer was extracted with ethyl acetate twice (2×100 mL). The combined organic layer was washed with brine (100 mL and concentrated to about two volumes. Two volumes of acetonitrile was added followed by 6 M HCI in isopropanol (100 mL) at 25±5° C. over a period of 10 minutes. The mixture was stirred for 2 hours at 25±5° C. to crystallize the product. The solid was filtered and washed with ethyl acetate-acetonitrile mixture (50 mL, 1:1 ratio) and dried the solid at 45° C. under vacuum for 5 hours to afford pure ethyl 3-amino-1H-pyrrole-2-carboxylate hydrochloride salt (40 g, 57% yield) as a pale-yellow solid.

Result: Input: 100 g, Output: 40 g, Isolated yield (input assay based): 57%, HPLC Purity: 97.7%.

¹ NMR (400MHz, DMSO-d₆ Free base): δ 12.13 (s, 1H), 9.74 (br s, 2H), 7.0 (t, 1H, J=2.8 Hz, 3.2 Hz), 6.23 (t, 1H, J=2.8 Hz), 4.27 (q, 2H, J=7.2 Hz), 1.3 (t, 3H, J=7.2 Hz).

Mass m/z (%): [M+H]⁺: 155.36 (free base).

Synthesis of 2-isopropoxyethyl methane sulfonate (VDP-e)

To a clean and dry 3 L flask equipped with mechanical overhead stirrer, thermometer socket, temperature probe, condenser and nitrogen inlet tube, was charged tert-butyl methyl ether (1.0 L, 10 vol.), triethyl amine (161.2 mL) and 2-isopropoxyethanol (100.0 g, 1.0 eq.). The reaction mass was cooled to 16° C. and to this was charged methanesulfonyl chloride (121.2 g, 1.1 eq.) and tert-butyl methyl ether (80 ml, 8 vol) slowly over a period of 2-3 hours while maintaining the temperature around 16° C. After addition, the reaction mass was stirred at 15-25° C. for 3 hours. The progress of reaction was monitored by GC (Note: Limit of 2-lsopropoxyethanol content is NMT 5.0%).

After completion of reaction, the reaction mixture was quenched with aq. sodium bicarbonate solution (30.0 g was dissolved in 600 mL water). The organic layer was separated and washed twice with water (2×400 mL, 8 vol.). The organic layer was separated and concentrated to minimum level (˜200 mL) at below 33° C. and twice with tert-butyl methyl ether (270 mL, 2.7 vol.) and again reduced to 200 mL. Then, tert-butyl methyl ether (100 mL) was added to prepare a 1:3 w/w solution of VDP-e (Note: The product was stored at cold conditions under temperature 4-8° C.).

Results: Input: 300 g, Output: 490 g, Isolated yield: 94%, HPLC Purity: 97%.

¹ NMR (400MHz, CDCl₃): δ 4.37 to 4.34 (m, 2H), 3.7 to 3.6 (m, 2H), 3.65 to 3.60 (m, 1H), 3.0 (s, 3H), 1.18 (d, 3H, J=5.6 Hz), 1.16 (d, 3H, J=5.6 Hz).

Mass m/z (%): [M+H]⁺: 182.23.

STEP (d): Synthesis of ethyl 3-((2-isopropoxyethyl) amino)-1H-pyrrole-2-carboxylate hydrochloride (VDP-f)

To a stirred solution of purified water (8 vol) with potassium carbonate (1.2 eq.) and potassium iodide (1.2 eq.) were added and stirred for 10 min to get clear solution, n-heptane (14 vol) was added and the solution was heated to 65° C. The first lot solution of 2-isopropoxyethyl mesylate in tert-butyl methyl ether (assay corrected, 1.5 eq.) was added to the mixture followed by continuous addition of a solution of ethyl 3-amino-1H-pyrrole-2-carboxylate hydrochloride salt (VDP-d) in water (1.0 eq. in 12 vol) over a period of 6 h. The remaining four lots of 2-isopropoxyethyl mesylate in tert-butyl methyl ether (0.375 eq.) were added at additional 2 h intervals. After completion of all lots maintained the reaction mixture for 18 hours at 65° C. The reaction was monitored by HPLC (Note: Limit of VDP-d content is NMT 5%).

After completion of reaction, the reaction mass was cooled to 25° C. The phases were separated and the aqueous phase was extracted twice with tert-butyl methyl ether (10 vol). The combined organic layer was washed with water (5 vol) and brine (5 vol). The organic phase was concentrated up to 5 volumes, then 6 N HCI in isopropanol at 25° C. was added and stirred for 1 hour and filtered the resulting solid and washed with MTBE and dried at 40° C. under vacuum for 4 hours.

Results: Input: 100 g, Output: 112 g, Isolated yield: 77%, HPLC Purity: 97.5%.

¹ NMR (HCI salt)(400MHz, CDCI₃) δ 11.79 (br. s, 1H), 8.7 (bs, 2H), 6.9 (bt, 1H, J=2.8 Hz), 6.1 (s, 1H), 4.26 to 4.21 (q, 2H, J=7.2 Hz), 3.59 to 3.51 (m, 3H), 3.32 to 3.29 (t, 2H, J=4.8 Hz), 1.3 to 1.27 (t, 3H, J=7.2 Hz), 1.2 (d, 6H, J=6.0 Hz) ppm.

Mass m/z (%) [M+H]⁺: 241.02 (free base).

STEP (f): Synthesis of ethyl 3-(3-benzoyl-1-(2-isopropoxyethyl) thioureido)-1H-pyrrole-2-carboxylate (VDP-g)

A stirred solution of ethyl 3-((2-isopropoxyethyl) amino)-1H-pyrrole-2-carboxylate hydrochloride (VDP-f) (1.0 eq.) and triethyl amine (1.1 eq.) in n-butyl acetate (5 vol) was cooled to 0° C. and benzoyl isothiocyanate (1.05 eq.) was added dropwise and the reaction temperature was raised to 25° C. and stirred for 3 h. The progress of the reaction was monitored by HPLC (Note: Limit of VDP-f content is NMT 1.0%). The mixture was then cooled again to 0° C. and n-heptane (10 vol) was slowly added to the reaction mass. The mixture was stirred at 0° C. for 3 h. The product was filtered off and the filtered cake was taken in water (3 vol) and stirred for 1 hour then filter the solid, washed with pre-cooled n-heptane (1 vol) and dried under vacuum in at 40° C. for 12 h (Note: LOD NMT 2%).

Result: Input: 112 g, Output: 138 g, isolated yield: 81%, HPLC Purity: 98.5%.

¹ NMR (400MHz, DMSO-d6) δ 11.77 (s, 1H), 10.38 (s, 1H), 7.49 to 7.45 (m, 3H), 7.36 to 7.33 (m, 2H), 6.81 to 6.80 (t, 1H, J=2.8 Hz), 6.15 to 6.13 (t, 1H, J=2.4 Hz), 4.28 to 4.16 (m, 4H), 3.75 to 3.73 (m, 1H), 3.56 to 3.45 (m, 2H), 1.33 to 1.29 (t, 3H, J=7.2 Hz), 1.02 to 0.99 (m, 6H).

Mass m/z (%) : [M+H]⁺: 404.18.

STEP 7: Synthesis of 1-(2-isopropoxyethyl)-2-thioxo-1,2,3,5-tetrahydro-4H-pyrrolo[3,2-d] pyrimidin-4-one (VDP-h)

To a clean and dry 2 L flask equipped with mechanical overhead stirrer, thermometer socket, temperature probe, condenser and nitrogen inlet tube, was charged methanol (800 ml, 8 vol), sodium hydroxide (14.84 g, 1.5 eq.) and stirred for 10 min. And then ethyl 3-(3-benzoyl-1-(2-isopropoxyethyl)thioureido)-1H-pyrrole-2-carboxylate (VDP-g) (100 g, 1.0 eq.) were added and heated to 60 to 65° C. and stirred for 4h. The reaction was monitored by HPLC (Note: Limit of VDP-g content is NMT 1.0%). After reaction is completed the temperature was brought to 25° C. Then slowly acetic acid (25.0 mL, 0.25 vol) was added to quench the reaction mass (Note: pH- 5.5 to 6.5). Then purified water (800 mL, 8 vol) was charged to the reaction mass over a period of 1h. The reaction mixture was cooled to 0-5° C. over a period of 2 h and stirred at 0-5° C. for 1 h. The product was filtered and washed twice with pre-cooled mixture of water (134 mL, 1.34 vol) and methanol (66 mL, 0.66 vol) and dried under vacuum at 40° C. (Limit: methanol content NMT 0.5%, water content NMT 3%) to afford 1-(2-isopropoxyethyl)-2-thioxo-1,2,3,5-tetrahydro-4H-pyrrolo[3,2-d] pyrimidin-4-one (VDP-h, verdiperstat) as an off-white solid.

Recrystallisation

The above off-white solid (VDP-h) was taken in isopropanol (14 vol) and heated to reflux (80 to 85° C.) to get a clear solution and then passed through carbon pads and 1-micron cartridge filter and rinsed the flask with isopropanol (1.0 vol), and collect the filtrate and stir for ambient temperature for 1.0 hour (VDP precipitated out). Then further cool to 0-5° C. and maintain for 1.0 hour. The product was filtered, washed with isopropanol (2 vol. pre-cooled to 0-5° C.) and dried under vacuum at 40° C. to afford 1-(2-isopropoxyethyl)-2-thioxo-1,2,3,5-tetrahydro-4H-pyrrolo[3,2-d] pyrimidin-4-one (VDP, BHV-3241) as an off-white solid. The product was analysed by HPLC, LC-MS, and H¹ NMR.

Result

Input: 100 g, Output: 58 g, Isolated yield: 92%, HPLC Purity: 99.8%.

¹ INMR (400MHz, DMSO-d₆) δ 12.35 (s, 1H), 12.18 (s, 1H), 7.3 (s, 1H), 6.27 (s, 1H), 4.48 to 4.45 (t, 2H, J=6.4 Hz), 3.72 to 3.69 (t, 2H, J=6.4 Hz), 3.57 to 3.51 (m, 1H), 1.0 to 0.98 (d, 6H, J=6.0 Hz).

Mass m/z (%) : [M+H]⁺: 254.31.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A process for preparing verdiperstat comprising the steps of:
reacting a compound having formula 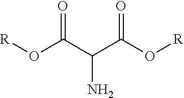
or a salt thereof, wherein R is the same or different and is each independently a C1-C5 alkyl, with 3-(dimethylamino)acrylonitrile to obtain a compound having formula 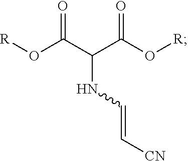
and
converting the compound having formula 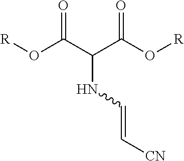
to verdiperstat.

2. The process according to claim 1, wherein the step of converting the compound having formula 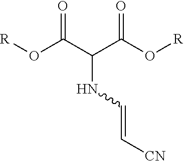
to verdiperstat comprises:
converting the compound having formula 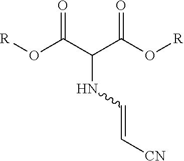
to a compound having formula 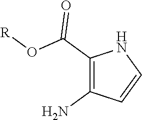
or a salt thereof; and
converting the compound having formula 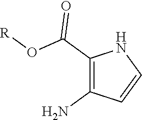
or a salt thereof to verdiperstat.

3. The process according to claim 1, wherein the step of reacting a compound having formula 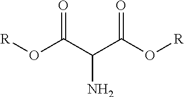
or a salt thereof, wherein R is a C1-C5 alkyl with 3-(dimethylamino)acrylonitrile is conducted in the presence of an acid.

4. The process according to claim 3, wherein the acid is a carboxylic acid.

5. The process according to claim 2, wherein the step of converting the compound having formula 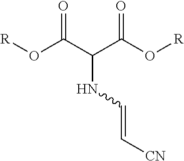
to a compound having formula 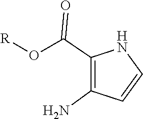
or a salt thereof is conducted in the presence of a base.

6. The process according to claim 5, wherein the base is a metal alkoxide.

7. The process according to claim 2, wherein the step of converting the compound having formula 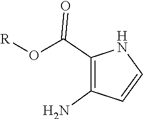
or a salt thereof to verdiperstat comprises reacting the compound having formula 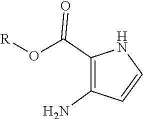
or a salt thereof with a compound having formula 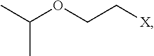
wherein X is a leaving group to obtain a compound having formula 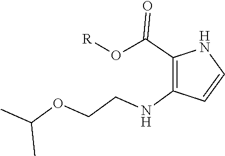
or a salt thereof.

8. The process according to claim 7, wherein the leaving group is an organic sulfonate group.

9. The process according to claim 7, wherein the step of reacting the compound having fomula 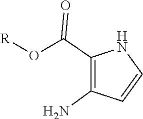
or a salt thereof with a compound having formula 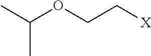
is conducted in the presence of a base, and optionally, a metal iodide.

10. The process according to claim 9, wherein the base is a salt of an inorganic acid, and wherein the metal is an alkali metal.

11. The process according to claim 7, wherein the step of converting the compound having formula 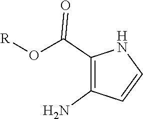
or a salt thereof to verdiperstat further comprises reacting the compound having formula 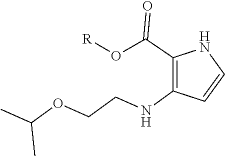
or a salt thereof with a compound having formula 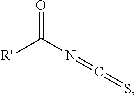
wherein R′ is H or an organic group, to obtain a compound having formula

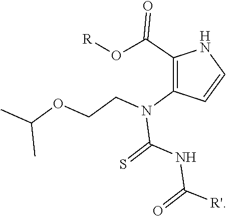

12. The process according to claim 7, wherein the step of converting the compound having formula 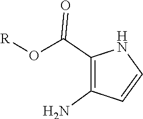
or a salt thereof to verdiperstat further comprises converting the compound having formula 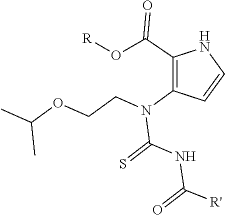
to verdiperstat.

13. The process according to claim 12, wherein the step of converting the compound having formula 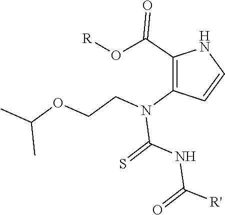
to verdiperstat is conducted in the presence of a base.

14. The process according to claim 13, wherein the base is a metal hydroxide.

15. The process according to claim 1, wherein the step of reacting a compound having formula 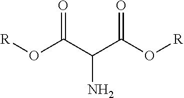
or a salt thereof, wherein R is a C1-C5 alkyl, with 3-(dimethylamino)acrylonitrile is preceded by reacting cyanoacetic acid with N, N-dimethylformamide dimethyl acetal to obtain 3-(dimethylamino)acrylonitrile.

16. A process for preparing verdiperstat comprising:
Step (a): reacting cyanoacetic acid with N, N-dimethylformamide dimethyl acetal to obtain 3-(dimethylamino)acrylonitrile;
Step (b): reacting a compound having formula 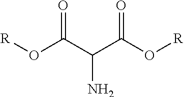
or a salt thereof,
wherein R is the same or different and is each independently a C1-C5 alkyl, with 3-(dimethylamino)acrylonitrile to obtain a compound having formula 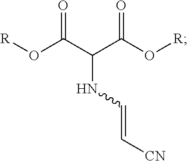
Step (c): converting the compound having formula 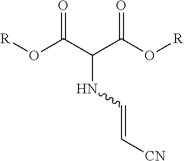
to a compound having formula 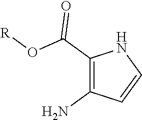
or a salt thereof;
Step (d): reacting the compound having formula 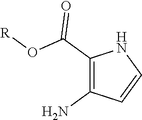
or a salt thereof with a compound having formula 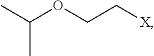
wherein X is a leaving group to obtain a compound having formula 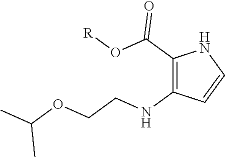
or a salt thereof;
Step (e): reacting the compound having formula 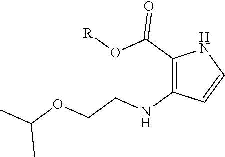
or a salt thereof with a compound having formula 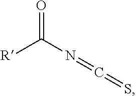
wherein R′ is H or an organic group, to obtain a
compound having formula 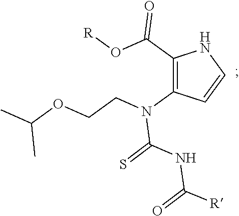
and
Step (f): converting the compound having formula 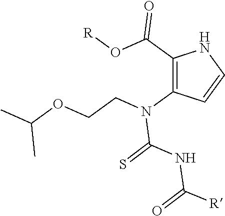
to verdiperstat.

17. A composition comprising:
a compound having formula 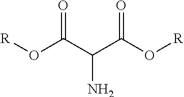
or a salt thereof, wherein R is the same or different and is each independently a C1-C5 alkyl; and
3-(dimethylamino)acrylonitrile.

18. The composition according to claim 17, further comprising a solvent, an acid, or a combination thereof.

19. The composition according to claim 18, wherein the solvent is a hydrocarbon solvent, a halogenated hydrocarbon solvent, or a combination thereof.

20. The composition according to claim 19, wherein the acid is a carboxylic acid.

21. A composition comprising verdiperstat and a compound having formula 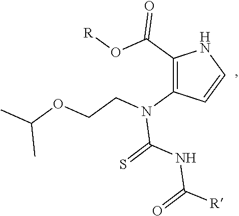
wherein R′ is H or an organic group.

22. The composition of claim 21, wherein the compound having formula 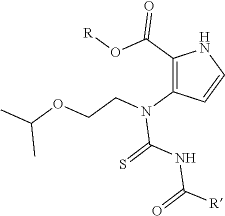
is present in an amount of 0.2 percent by weight or greater based on the total weight of the composition.

23. The composition of claim 21, wherein the compound having formula 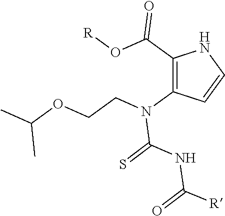
is present in an amount of less than 0.2 percent by weight based on the total weight of the composition.
